(19) United States
(12) Patent Application Publication
(10) Pub. No.: US 2026/0116977 A1
(43) Pub. Date: Apr. 30, 2026

(54) METHODS OF TREATING MELANOMA USING AN ANTI-CTLA4 ANTIBODY

(71) Applicant: AGENUS INC., Lexington, MA (US)

(72) Inventors: Jaymin Patel, Lexington, MA (US); Steven O'Day, Lexington, MA (US); Dhan Sidhartha Chand, Lexington, MA (US)

(21) Appl. No.: 19/341,862

(22) Filed: Sep. 26, 2025

Related U.S. Application Data
(63) Continuation of application No. PCT/US2024/021931, filed on Mar. 28, 2024.
(60) Provisional application No. 63/492,717, filed on Mar. 28, 2023.

(51) Int. Cl. C07K 16/28; A61K 9/00; A61K 39/00; A61P 35/00

(52) U.S. Cl. CPC C07K 16/2818; A61K 9/0019; A61P 35/00; A61K 2039/505; A61K 2039/54; A61K 2039/545; C07K 2317/52; C07K 2317/565

(57) ABSTRACT

Provided are methods for treating melanoma with an antibody that specifically binds to human Cytotoxic T-Lymphocyte Antigen 4 (CTLA-4).

### RELATED APPLICATIONS

This application is a continuation of International Application Serial No. PCT/US2024/021931, filed Mar. 28, 2024, which claims priority to U.S. Provisional Patent Application Ser. No. 63/492,717, filed Mar. 28, 2023, the entire disclosures of which are hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Sep. 26, 2025, is named “220529_seqlist.xml” and is 10,382 bytes in size).

### BACKGROUND

Melanoma is a lethal form of skin cancer whose incidence has been rising rapidly over recent decades. Although melanoma accounts for only 1% of skin cancers, most skin cancer deaths are caused by melanoma because of its ability to metastasize.

Late-stage melanomas are hard to treat but in recent years, immunotherapies have shown promising results in patients with stage III or stage IV melanoma. Currently, anti-PD-1 antibody monotherapy or anti-PD-1 antibody/anti-CTLA-4 antibody combination therapy are approved for first line treatment in patients with stage III or stage IV melanoma. However, approximately 50% of patients with metastatic melanoma fail to achieve an objective response with these treatments and of those who do respond, many subsequently relapse.

Accordingly, alternative immunotherapy treatment options for patients who progress on first-line anti-PD-1 antibody monotherapy or first-line anti-PD-1/anti-CTLA-4 combination therapy are needed.

### SUMMARY

The instant disclosure is directed to methods for treating melanoma with an antibody that specifically binds to human Cytotoxic T-Lymphocyte Antigen 4 (CTLA-4). Also provided herein are particular methods for administering an antibody that specifically binds to human CTLA-4 that results in a reduction of tumor burden in a subject. In an embodiment, the method comprises a therapeutically effective amount that safely and effectively treats melanoma. The methods disclosed herein are not limited to the treatment of stage III or stage IV melanoma, and thus can be used to treat subjects who have other stages of melanoma, e.g., stage I or stage II.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has melanoma, the method comprising administering to the subject an antibody that specifically binds to human CTLA-4 at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody that specifically binds to human CTLA-4 is administered at a dose of 50 mg to 175 mg. In an embodiment, the antibody that specifically binds to human CTLA-4 is administered at a dose of 75 mg to 150 mg. In an embodiment, the antibody that specifically binds to human CTLA-4 is administered at a dose of 25 mg, 50 mg, 75 mg, 100 mg, or 150 mg.

In an embodiment, the antibody that specifically binds to human CTLA-4 is administered intravenously. In an embodiment, the antibody that specifically binds to human CTLA-4 is administered by intravenous infusion over 30 minutes.

In an embodiment, the antibody that specifically binds to human CTLA-4 is administered once weekly. In an embodiment, the antibody that specifically binds to human CTLA-4 is administered once every 2 weeks. In an embodiment, the antibody that specifically binds to human CTLA-4 is administered once every 3 weeks. In an embodiment, the antibody that specifically binds to human CTLA-4 is administered once every 4 weeks. In an embodiment, the antibody that specifically binds to human CTLA-4 is administered once every 5 weeks. In an embodiment, the antibody that specifically binds to human CTLA-4 is administered once every 6 weeks.

In an embodiment, the antibody that specifically binds to human CTLA-4 is administered intravenously at a dose of 25 mg once every 3 weeks. In an embodiment, the antibody that specifically binds to human CTLA-4 is administered intravenously at a dose of 50 mg once every 3 weeks. In an embodiment, the antibody that specifically binds to human CTLA-4 is administered intravenously at a dose of 75 mg once every 3 weeks. In an embodiment, the antibody that specifically binds to human CTLA-4 is administered intravenously at a dose of 100 mg once every 3 weeks. In an embodiment, the antibody that specifically binds to human CTLA-4 is administered intravenously at a dose of 150 mg once every 3 weeks.

In an embodiment, the dose is a therapeutically effective amount.

In an embodiment, the melanoma is cutaneous melanoma. In an embodiment, the melanoma is unresectable. In an embodiment, the melanoma is metastatic. In an embodiment, the melanoma is ocular melanoma, uveal melanoma, or mucosal melanoma.

In an embodiment, the melanoma is refractory to a checkpoint inhibitor therapy. In an embodiment, the checkpoint inhibitor therapy is an anti-PD-1 antibody, an anti-PD-L1 antibody, ipilimumab, or tremelimumab.

In an embodiment, the subject has received at least one prior anti-cancer therapy. In an embodiment, the at least one prior anti-cancer therapy is an anti-PD-1 antibody, optionally wherein the anti-PD-1 antibody is balstilimab, nivolumab, or pembrolizumab; an anti-PD-L1 antibody; ipilimumab; or tremelimumab.

In an embodiment, the at least one prior anti-cancer therapy is a BRAF inhibitor and/or a MEK inhibitor. In an embodiment, the BRAF inhibitor is selected from the group consisting of vemurafenib, dabrafenib, and encorafenib. In an embodiment, the MEK inhibitor is selected from the group consisting of trametinib, cobimetinib, and binimetinib.

In an embodiment, the antibody that specifically binds to human CTLA-4 is administered to the subject prior to radiotherapy, a chemotherapy, or surgical removal of a tumor.

In an embodiment, the subject has not had prior anti-CTLA-4 antibody therapy.

In an embodiment, the subject has unresectable Stage III or Stage IV cutaneous melanoma.

In an embodiment, the subject has a BRAF mutation. In an embodiment, the BRAF mutation is a BRAF V600 mutation.

In an embodiment, the subject is homozygous for phenylalanine in position 158 of FcγRIIIA. In an embodiment, the subject is homozygous for valine in position 158 of FcγRIIIA. In an embodiment, the subject is heterozygous for valine/phenylalanine in position 158 of FcγRIIIA.

In an embodiment, the melanoma is not ocular melanoma. In an embodiment, the melanoma is not uveal melanoma. In an embodiment, the melanoma is not mucosal melanoma.

In an embodiment, the subject does not have any persistent toxicities (Common Terminology Criteria for Adverse Events [CTCAE]≥2) from prior cancer therapies.

In an embodiment, the subject does not have any history of CTCAE≥3 immune-mediated toxicity (excluding endocrinopathies and non-necrotizing/bullous rash) from prior checkpoint inhibition.

In an embodiment, administration of the antibody reduces tumor size in the subject. In an embodiment, administration of the antibody increases T-cell activation in the subject.

In an embodiment, before administration of the antibody the subject has measurable disease on baseline imaging per RECIST 1.1. In an embodiment, before administration of the antibody the subject has an Eastern Cooperative Oncology Group performance status (PS) 0-1. In an embodiment, before administration of the antibody the subject has a predicted life expectancy of ≥3 months.

In an embodiment, before administration of the antibody the subject has: adequate organ function as defined by one or more of: a) neutrophils≥1500/μL; b) platelets≥100× 10³/μL; c) hemoglobin≥8.0 g/dL; d) creatinine clearance≥45 mL/min as measured or calculated per local institutional standards; e) AST/ALT≤3×upper limit of normal (ULN); f) total bilirubin≤1.5×ULN (except patients with Gilbert syndrome who must have a total bilirubin level of ≤3.0×ULN); g) albumin≥3.0 g/dL; and/or h) International normalized ratio or prothrombin time≤1.5×ULN and activated partial thromboplastin time≤1.5×ULN (unless patient is receiving anticoagulant therapy).

In an embodiment, the subject does not have partial or complete bowel obstruction within the last 3 months, signs/symptoms of bowel obstruction, or known radiologic evidence of impending obstruction.

In an embodiment, the subject does not have refractory ascites defined as requiring 2 or more therapeutic paracentesis in the last 4 weeks or ≥4 within the last 90 days prior to administration of the antibody.

In an embodiment, the subject does not have clinically significant cardiovascular disease.

In an embodiment, the subject does not have active brain metastases or leptomeningeal metastases. In an embodiment, the subject does not have a concurrent malignancy that requires treatment or a history of prior malignancy that was active within 2 years prior to administration of the antibody.

In an embodiment, the subject has not had a cytotoxic therapy or targeted therapy, within 3 weeks prior to administration of the antibody. In an embodiment, the subject has not had other monoclonal antibody therapy, antibody-drug conjugate therapy, or radioimmunoconjugate therapy, within 4 weeks prior to administration of the antibody.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of 50 mg once every 3 weeks, wherein the subject has stage III or stage IV cutaneous melanoma, and wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of 150 mg once every 3 weeks, wherein the subject has stage III or stage IV cutaneous melanoma, and wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody that specifically binds to human CTLA-4 comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody that specifically binds to human CTLA-4 comprises a VH comprising the amino acid sequence set forth in SEQ ID NO: 7 and a VL comprising the amino acid sequence set forth in SEQ ID NO: 8.

In an embodiment, the antibody that specifically binds to human CTLA-4 comprises a human IgG1 heavy chain constant region comprising S239D/A330L/1332E mutations, numbered according to the EU numbering system.

In an embodiment, the antibody that specifically binds to human CTLA-4 comprises a heavy chain comprising the amino acid sequence set forth in SEQ ID NO: 9 and a light chain comprising the amino acid sequence set forth in SEQ ID NO: 10.

In an embodiment, the antibody that specifically binds to human CTLA-4 is botensilimab.

In an aspect, provided herein is an antibody that specifically binds to human CTLA-4 for use in the treatment of melanoma, wherein the treatment is performed according to the method of any one of the previous claims.

In an aspect, provided herein is an antibody that specifically binds to human CTLA-4 for use in the manufacture of a medicament for the treatment of melanoma, wherein the treatment is performed according to the method of any one of the previous claims.

In an aspect, provided herein is a use of an antibody that specifically binds to human CTLA-4 for the treatment of melanoma, wherein the treatment is performed according to the method of any one of the previous claims.

### BRIEF DESCRIPTION OF THE DRAWINGS

FIG. 1A and FIG. 1B show the percent change (FIG. 1A) and best percent change (FIG. 1B) of tumor burden from baseline over time, in 10 cutaneous melanoma patients that received botensilimab as monotherapy or in combination with balstilimab, according to aspects of the present disclosure.

### DETAILED DESCRIPTION

The instant disclosure is directed to methods for treating melanoma with an antibody that specifically binds to human Cytotoxic T-Lymphocyte Antigen 4 (CTLA-4). Also provided herein are particular methods for administering an antibody that specifically binds to human CTLA-4 that results in a reduction of tumor burden in a subject. In an embodiment, the method comprises a therapeutically effective amount that safely and effectively treats melanoma.

### Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In an embodiment, the antibody is a humanized monoclonal antibody. In an embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of Proteins of Immunological Interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other (see Table 1 below). In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of Proteins of Immunological Interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human CTLA-4). CDRH1, CDRH2, and CDRH3 denote the heavy chain CDRs, and CDRL1, CDRL2, and CDRL3 denote the light chain CDRs.

TABLE 1
CDR definitions

| | CDR Definitions | | |
|---|---|---|---|
| | Kabat | Chothia | MacCallum |
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen.

As used herein, the term “EU numbering system” refers to the EU numbering convention for the constant regions of an antibody, as described in Edelman G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “subject” includes any human or non-human animal. In an embodiment, the subject is a human.

As used herein, the term “effective amount” in the context of the administration of a therapy to a subject refers to the amount of a therapy that achieves a desired prophylactic or therapeutic effect.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “targeted therapy” refers to a therapy that inhibits a specific protein. In an embodiment, the targeted therapy inhibits a protein that is known to be important for growth and/or survival of melanoma cells (e.g., BRAF).

As used herein, the term “cytotoxic therapy” refers to a therapy that blocks or slows cell division. In an embodiment, the cytotoxic therapy kills cancer cells. In an embodiment, the cytotoxic therapy is fluorouracil, capecitabine, oxaliplatin, irinotecan, or trifluridine-tipiracil.

As used herein, the term “tumor burden” refers to the number of cancer cells, the size of a tumor, or the amount of cancer in the body of the subject.

As used herein, the term “about” when referring to a measurable value, such as a dosage, encompasses variations of +20%, +15%, +10%, +5%, +1%, or +0.1% of a given value or range, as are appropriate to perform the methods disclosed herein.

### Anti-CTLA-4 Antibodies

Antibodies that specifically bind to human CTLA-4 (i.e., anti-CTLA-4 antibodies) that are useful in the methods and uses described herein include but are not limited to those listed below.

In an embodiment, the antibody comprises: a heavy chain variable region (VH) comprising the CDRH1, CDRH2, and CDRH3 amino acid sequences of the VH amino acid sequence set forth in SEQ ID NO: 7. In an embodiment, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, and CDRH3 amino acid sequences set forth in SEQ ID NO: 1, 2, and 3, respectively. In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7. In an embodiment, the antibody comprises: a VH comprising the amino acid sequence set forth in SEQ ID NO: 7; and a VL comprising an amino acid sequence which is at least 90%, 95%, 96%, 97%, 98%, 99%, or 100% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a heavy chain constant region selected from the group consisting of human IgG1, IgG2, IgG3, IgG4, IgA1, and IgA2. In an embodiment, the heavy chain constant region is IgG1. In an embodiment, the heavy chain constant region is IgG2. In an embodiment, the antibody comprises a light chain constant region selected from the group consisting of a human kappa light chain constant region and a human lambda light chain constant region.

In an embodiment, the antibody comprises an IgG1 heavy chain constant region. In an embodiment, the amino acid sequence of the IgG1 heavy chain constant region comprises S239D/1332E mutations, numbered according to the EU numbering system. In an embodiment, the amino acid sequence of the IgG₁ heavy chain constant region comprises S239D/A330L/1332E mutations, numbered according to the EU numbering system. In an embodiment, the amino acid sequence of the IgG₁ heavy chain constant region comprises L235V/F243L/R292P/Y300L/P396L mutations, numbered according to the EU numbering system. In an embodiment, the IgG₁ heavy chain constant region is afucosylated IgG₁.

In an embodiment, the antibody comprises a heavy chain comprising the amino acid sequence set forth in SEQ ID NO: 9. In an embodiment, the antibody comprises a heavy chain comprising the amino acid sequence set forth in SEQ ID NO: 9 and a light chain comprising an amino acid sequence which is at least 90%, 95%, 96%, 97%, 98%, 99%, or 100% identical to the amino acid sequence set forth in SEQ ID NO: 10.

In an embodiment, the antibody comprises a heavy chain comprising the amino acid sequence set forth in SEQ ID NO: 9 and a light chain comprising the amino acid sequence set forth in SEQ ID NO: 10. In an embodiment, the amino acid sequence of the heavy chain consists of the amino acid sequence set forth in SEQ ID NO: 9 and the amino acid sequence of the light chain consists of the amino acid sequence set forth in SEQ ID NO: 10.

In an embodiment the antibody is botensilimab (a.k.a. AGEN1181), the amino acid sequences of which are provided in Table 2 below.

TABLE 2
Amino acid sequences of botensilimab

| | SEQ ID | |
|---|---|---|
| Description | NO: | Amino acid sequence |
| CDRH1 | 1 | SYSMN |
| CDRH2 | 2 | SISSSSSYIYYAESVKG |
| CDRH3 | 3 | VGLFGPFDI |
| CDRL1 | 4 | RASQSVSRYLG |
| CDRL2 | 5 | GASTRAT |
| CDRL3 | 6 | QQYGSSPWT |
| VH | 7 | EVQLVESGGGLVKPGGSLRLSCAASGFTFSSYSMNWVRQ |
| | | APGKGLEWVSSISSSSSYIYYAESVKGRFTISRDNAKNSLY |
| | | LQMNSLRAEDTAVYYCARVGLFGPFDIWGQGTLVTVSS |
| VL | 8 | EIVLTQSPGTLSLSPGERATLSCRASQSVSRYLGWYQQKP |
| | | GQAPRLLIYGASTRATGIPDRFSGSGSGTDFTLTITRLEPE |
| | | DFAVYYCQQYGSSPWTFGQGTKVEIK |
| Heavy chain | 9 | EVQLVESGGGLVKPGGSLRLSCAASGFTFSSYSMNWVRQ |
| | | APGKGLEWVSSISSSSSYIYYAESVKGRFTISRDNAKNSLY |
| | | LQMNSLRAEDTAVYYCARVGLFGPFDIWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKRVEPKSCDKTHTCPPCPAPELLG |
| | | GPDVFLFPPKPKDTLMISRTPEVTCVVVDVSHEDPEVKEN |
| | | WYVDGVEVHNAKTKPREEQYNSTYRVVSVLTVLHQDW |
| | | LNGKEYKCKVSNKALPLPEEKTISKAKGQPREPQVYTLPP |
| | | SREEMTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPG |
| Light chain | 10 | EIVLTQSPGTLSLSPGERATLSCRASQSVSRYLGWYQQKP |
| | | GQAPRLLIYGASTRATGIPDRESGSGSGTDFTLTITRLEPE |
| | | DFAVYYCQQYGSSPWTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |

### Methods of Treatment

The instant disclosure demonstrates that antibodies that specifically bind to human CTLA-4 (e.g., botensilimab) are highly effective in treating melanoma. The instant disclosure also demonstrates that antibodies that specifically bind to human CTLA-4 (e.g., botensilimab) are highly effective in treating stage III or stage IV cutaneous melanoma that is refractory to a checkpoint inhibitor therapy (e.g., anti-PD-1 antibody, anti-PD-L1 antibody). Accordingly, the instant disclosure is broadly directed to methods for treating melanoma with an antibody that specifically binds to human CTLA-4.

In an aspect, provided herein is a method of treating melanoma in a subject in need thereof, the method comprising administering to the subject an effective amount of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has melanoma, the method comprising administering to the subject an effective amount of an antibody that specifically binds to human CTLA-4, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of about 5 mg to about 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has melanoma, the method comprising administering to the subject an antibody that specifically binds to human CTLA-4 at a dose of about 5 mg to about 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of 5 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of enhancing the activation of T cells in a subject who has melanoma, the method comprising administering to the subject an antibody that specifically binds to human CTLA-4 at a dose of 5 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody is administered at a dose of about 25 mg to about 200 mg. In an embodiment, the antibody is administered at a dose of about 25 mg to about 150 mg. In an embodiment, the antibody is administered at a dose of about 50 mg to about 150 mg. In an embodiment, the antibody is administered at a dose of about 75 mg to about 150 mg.

In an embodiment, the antibody is administered at a dose of 25 mg to 200 mg. In an embodiment, the antibody is administered at a dose of 50 mg to 175 mg. In an embodiment, the antibody is administered at a dose of 25 mg to 150 mg. In an embodiment, the antibody is administered at a dose of 50 mg to 150 mg. In an embodiment, the antibody is administered at a dose of 75 mg to 150 mg.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, or about 200 mg.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, or 200 mg.

In an embodiment, the antibody is administered intravenously. In an embodiment, the antibody is administered intratumorally.

In an embodiment, the antibody is administered by intravenous infusion over about 30 minutes. In an embodiment, the antibody is administered by intravenous infusion over about 45 minutes. In an embodiment, the antibody is administered by intravenous infusion over about 60 minutes. In an embodiment, the antibody is administered by intravenous infusion over about 90 minutes.

In an embodiment, the antibody is administered about once weekly. In an embodiment, the antibody is administered about once every 2 weeks. In an embodiment, the antibody is administered about once every 3 weeks. In an embodiment, the antibody is administered about once every 4 weeks. In an embodiment, the antibody is administered about once every 5 weeks. In an embodiment, the antibody is administered about once every 6 weeks. In an embodiment, the antibody is administered about once every 7 weeks. In an embodiment, the antibody is administered about once every 8 weeks.

In an embodiment, the antibody is administered once weekly. In an embodiment, the antibody is administered once every 2 weeks. In an embodiment, the antibody is administered once every 3 weeks. In an embodiment, the antibody is administered once every 4 weeks. In an embodiment, the antibody is administered once every 5 weeks. In an embodiment, the antibody is administered once every 6 weeks. In an embodiment, the antibody is administered once every 7 weeks. In an embodiment, the antibody is administered once every 8 weeks.

In an embodiment, the antibody is administered intravenously at a dose of about 25 mg once every 3 weeks. In an embodiment, the antibody is administered intravenously at a dose of about 50 mg once every 3 weeks. In an embodiment, the antibody is administered intravenously at a dose of about 75 mg once every 3 weeks. In an embodiment, the antibody is administered intravenously at a dose of about 100 mg once every 3 weeks. In an embodiment, the antibody is administered intravenously at a dose of about 150 mg once every 3 weeks.

In an embodiment, the antibody is administered intravenously at a dose of 25 mg once every 3 weeks. In an embodiment, the antibody is administered intravenously at a dose of 50 mg once every 3 weeks. In an embodiment, the antibody is administered intravenously at a dose of 75 mg once every 3 weeks. In an embodiment, the antibody is administered intravenously at a dose of 100 mg once every 3 weeks. In an embodiment, the antibody is administered intravenously at a dose of 150 mg once every 3 weeks.

In an embodiment, the dose is a therapeutically effective amount.

In an embodiment, the melanoma is cutaneous melanoma. In an embodiment, the melanoma is ocular melanoma, uveal melanoma, or mucosal melanoma. In an embodiment, the melanoma is unresectable. In an embodiment, the melanoma is metastatic. In an embodiment, the melanoma is relapsed and/or refractory.

In an embodiment, the melanoma is refractory to a checkpoint inhibitor therapy. In an embodiment, the checkpoint inhibitor therapy is an anti-PD-1 antibody, an anti-PD-L1 anti-body, ipilimumab, or tremelimumab.

In an embodiment, the subject has received at least one prior anti-cancer therapy. In an embodiment, the at least one prior anti-cancer therapy is an anti-PD-1 antibody, optionally wherein the anti-PD-1 antibody is balstilimab, nivolumab, or pembrolizumab; an anti-PD-L1 antibody; ipilimumab; or tremelimumab. In an embodiment, the at least one prior anti-cancer therapy is a BRAF inhibitor and/or a MEK inhibitor. In an embodiment, the BRAF inhibitor is selected from the group consisting of vemurafenib, dabrafenib, and encorafenib. In an embodiment, the MEK inhibitor is selected from the group consisting of trametinib, cobimetinib, and binimetinib. In an embodiment, the subject has received at least one prior anti-cancer therapy selected from the group consisting of an anti-PD-1 antibody (optionally wherein the anti-PD-1 antibody is balstilimab, nivolumab, or pembrolizumab), an anti-PD-L1 antibody, ipilimumab, tremelimumab, a BRAF inhibitor, or a MEK inhibitor.

In an embodiment, the antibody that specifically binds to human CTLA-4 is administered to the subject prior to radiotherapy, a chemotherapy, or surgical removal of a tumor.

In an embodiment, the subject has not had prior anti-CTLA-4 antibody therapy. In an embodiment, the subject has not previously been treated with an anti-CTLA-4 antibody with a modified Fc (e.g., BMS-96218, BMS-986288, HBM4003, XTX101).

In an embodiment, the subject has unresectable Stage III or Stage IV cutaneous melanoma.

In an embodiment, the subject has a BRAF mutation. In an embodiment, the BRAF mutation is a BRAF V600 mutation.

In an embodiment, the subject is homozygous for phenylalanine in position 158 of FcγRIIIA. In an embodiment, the subject is homozygous for valine in position 158 of FcγRIIIA. In an embodiment, the subject is heterozygous for valine/phenylalanine in position 158 of FcγRIIIA. Methods to assess the allele status of the FcγRIIIA gene are readily available and known to those of skill in the art. For example, without limitation, high-speed sequencing, DNA microarrays, and PCR amplification specific for the FcγRIIIA gene can be performed on tissue and/or blood samples collected from the subject.

In an embodiment, the melanoma is not ocular melanoma. In an embodiment, the melanoma is not uveal melanoma. In an embodiment, the melanoma is not mucosal melanoma.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of 50 mg once every 3 weeks, wherein the subject has stage III or stage IV cutaneous melanoma, and wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an aspect, provided herein is a method of treating melanoma in a subject in need thereof, the method comprising administering to the subject an antibody that specifically binds to human Cytotoxic T-Lymphocyte Antigen 4 (CTLA-4) at a dose of 150 mg once every 3 weeks, wherein the subject has stage III or stage IV cutaneous melanoma, and wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the subject does not have any persistent toxicities (Common Terminology Criteria for Adverse Events [CTCAE]≥2) from prior cancer therapies.

In an embodiment, the subject does not have any history of CTCAE≥3 immune-mediated toxicity (excluding endocrinopathies and non-necrotizing/bullous rash) from prior check-point inhibition.

In an embodiment, before administration of the antibody the subject has measurable disease on baseline imaging per RECIST 1.1. In an embodiment, before administration of the antibody the subject has an Eastern Cooperative Oncology Group performance status (PS) 0-1. In an embodiment, before administration of the antibody the subject has a predicted life expectancy of ≥12 weeks.

In an embodiment, before administration of the antibody the subject has: adequate organ function as defined by one or more of: a) neutrophils≥1500/μL; b) platelets≥100× 10³/μL; c) hemoglobin≥8.0 g/dL; d) creatinine clearance≥45 mL/min as measured or calculated per local institutional standards; e) AST/ALT≤3×upper limit of normal (ULN); f) total bilirubin≤1.5×ULN (except patients with Gilbert syndrome who must have a total bilirubin level of ≤3.0×ULN); g) albumin≥3.0 g/dL; and/or h) International normalized ratio or prothrombin time≤1.5×ULN and activated partial thromboplastin time≤1.5×ULN (unless patient is receiving anticoagulant therapy).

In an embodiment, the subject does not have partial or complete bowel obstruction within the last 3 months, signs/symptoms of bowel obstruction, or known radiologic evidence of impending obstruction.

In an embodiment, the subject has not received an immune checkpoint inhibitor therapy prior to administration of the antibody. In an embodiment, the subject has not received more than one chemotherapy regimen prior to administration of the antibody. In an embodiment, the subject does not have active brain metastases. In an embodiment, the subject does not have a concurrent malignancy that requires treatment or a history of prior malignancy that was active within 2 years prior to administration of the antibody. In an embodiment, the subject has not received a cytotoxic therapy or targeted therapy, within 3 weeks prior to administration of the antibody. In an embodiment, the subject has not received other monoclonal antibody therapy, antibody-drug conjugate therapy, or radioimmunoconjugate therapy, within 4 weeks prior to administration of the antibody.

In an embodiment, the subject does not have refractory ascites defined as requiring 2 or more therapeutic paracenteses within the last 4 weeks or ≥4 times within the last 90 days or ≥1 time within the last 2 weeks prior to administration of the antibody. In an embodiment, the subject does not have clinically significant cardiovascular disease.

In an embodiment, the objective response rate (ORR), duration of response (DOR), disease control rate (DCR), and progression-free survival (PFS) are assessed for a subject according to the Response Evaluation Criteria in Solid Tumors Version 1.1 (RECIST 1.1).

In an embodiment, the method results in a complete response, as defined by RECIST 1.1. In an embodiment, the method results in a partial response, as defined by RECIST 1.1. In an embodiment, the method results in a stable disease, as defined by RECIST 1.1.

In an embodiment, the method results in about a 1, 5, 10, 20, 30, 40, 50, 60, 70, 80, 90, or 100% reduction in tumor burden in the subject. In an embodiment, the method results in no change in tumor burden in the subject. In an embodiment, the method results in about a 1% reduction in tumor burden in the subject. In an embodiment, the method results in about a 5% reduction in tumor burden in the subject. In an embodiment, the method results in about a 10% reduction in tumor burden in the subject. In an embodiment, the method results in about a 20% reduction in tumor burden in the subject. In an embodiment, the method results in about a 30% reduction in tumor burden in the subject. In an embodiment, the method results in about a 40% reduction in tumor burden in the subject. In an embodiment, the method results in about a 50% reduction in tumor burden in the subject. In an embodiment, the method results in about a 60% reduction in tumor burden in the subject. In an embodiment, the method results in about a 70% reduction in tumor burden in the subject. In an embodiment, the method results in about an 80% reduction in tumor burden in the subject. In an embodiment, the method results in about a 90% reduction in tumor burden in the subject. In an embodiment, the method results in about a 100% reduction in tumor burden in the subject.

In an embodiment, the method results in a reduced tumor burden. In an embodiment, the method results in increased survival. In an embodiment, the method results in an increase in overall survival. In an embodiment, the method results in an increase in progression-free survival.

In an embodiment, any one of the methods disclosed herein further comprises administering an additional therapeutic agent to the subject. In an embodiment, the additional therapeutic is an antibody that specifically binds to human PD-1. In an embodiment, the additional therapeutic is balstilimab.

In an aspect, provided herein is an antibody that specifically binds to human CTLA-4 for use in the treatment of melanoma, wherein the treatment is performed according to the meth-od of any one of the previous claims.

In an aspect, provided herein is an antibody that specifically binds to human CTLA-4 for use in the manufacture of a medicament for the treatment of melanoma, wherein the treatment is performed according to the method of any one of the previous claims.

In an aspect, provided herein is a use of an antibody that specifically binds to human CTLA-4 for the treatment of melanoma, wherein the treatment is performed according to the method of any one of the previous claims.

### EXAMPLES

### Example 1—Multicohort, Open Label, Phase 2 Study of Botensilimab (AGEN1181) for Treatment of Advanced Melanoma Refractory to Prior Checkpoint Inhibitor Therapy

This Phase 2 study evaluated the clinical efficacy and safety of botensilimab as monotherapy and in combination with balstilimab in patients with advanced melanoma refractory to anti-PD-(L) 1-based therapy and in patients with advanced melanoma refractory to anti-PD-(L) 1 and anti-CTLA-4 combination therapy.

### A. Study Design

### Overall Design

This was a multicenter, multicohort, open-label Phase 2 trial of botensilimab in patients with advanced melanoma refractory to checkpoint inhibitor therapy.

Patients had histological confirmation of Stage III (unresectable) or Stage IV cutaneous melanoma, as per the American Joint Committee on Cancer (AJCC) 8th edition staging system. Baseline study procedures included cross-sectional imaging of the chest, abdomen and pelvis, (C/A/P) magnetic resonance imaging (MRI) of brain, routine laboratory testing and tumor markers, i.e., lactate dehydrogenase (LDH). Archival tumor tissue and/or fresh tissue as well as whole blood was collected at baseline for correlative studies.

Approximately 220 evaluable patients were enrolled. The study consisted of two parts.

In Part 1, a minimum of 60 patients each in Cohort A and Cohort B were randomized 1:1 (unless an arm was closed early for futility/toxicity or enrollment extended beyond 30 patients) to 1 of 2 dose levels (Arm 1 and Arm 2) of botensilimab as described below. Patients in Part 1 (in all arms) received botensilimab for up to 4 cycles or until death, unacceptable toxicity, loss to follow-up, disease progression, withdrawal from the stdy, or the Sponsor closes the study, whichever occurs first.
Cohort A (patients refractory to PD-(L) 1). Arm 1: botensilimab 50 mg IV Q3W for up to 4 cycles; Arm 2: botensilimab 150 mg IV Q3W for up to 4 cycles.
Cohort B (patients refractory to PD-(L) 1 and CTLA-4). Arm 1: botensilimab 50 mg IV Q3W for up to 4 cycles; Arm 2: botensilimab 150 mg IV Q3W for up to 4 cycles.

In Part 2, a minimum of 50 additional patients each in Cohort A and Cohort B received botensilimab in combination with balstilimab as described below.
Cohort A (patients refractory to PD-(L) 1). Arm 3: botensilimab 75 mg Q3W+balstilimab 450 mg Q3W; Arm 4: botensilimab 75 mg Q3W+balstilimab 450 mg Q3W.

Patients in Part 1 were stratified by prior BRAF therapy and LDH levels. Additional dose cohorts may be opened at the Sponsor's discretion based on emerging data.

TABLE 3
Study Treatments

| Study Treatment Name: | Botensilimab | Balstilimab |
|---|---|---|
| Dosage Formulation: | Solution for infusion | Solution for infusion |
| Unit Dosage Strength: | 50 mg/mL | 50 mg/mL |
| Dosage Level: | 50, 75, or 150 mg | 450 mg |
| Route of Administration: | intravenous (IV) | intravenous (IV) |
| | infusion | infusion |

During the treatment period, patients had routine clinical visits for administration of study treatment and safety monitoring.

Anti-tumor efficacy was determined via imaging assessments, which were performed every 9 weeks (Q9W) for the first year (through Week 54) and then every 12 weeks (Q12W). Disease response evaluation was per the investigator but may have been evaluated by independent central review per the Sponsor discretion.

Safety was assessed via monitoring of adverse event (AE), serious adverse event (SAE), treatment discontinuation due to AE, physical examinations, vital signs, hematology and chemistry laboratories. Patients who discontinued study treatment for reasons other than progressive disease continued with imaging assessments. For Survival Follow-up, patients who began additional anti-neoplastic therapy, or who have discontinued the study for other reasons, were followed Q12W for survival status via telephone call.

The two cohorts were evaluated separately in statistical plans.

### Study Drug Administration

Botensilimab was administered via IV infusion over 30 (+5) minutes. Patients were observed for 1 hour after the end of the infusion for infusion related reactions for the first 2 cycles and for 30 minutes after the end of each infusion thereafter. Measurement of vital signs was performed at each cycle prior to starting each infusion and at the end of each infusion. Infusions were followed immediately with a saline flush of the IV line, per institutional guidelines.

In the combination arm, balstilimab was administered prior to botensilimab. Both balstilimab and botensilimab were administered via IV infusion over 30 (+5) minutes.

### Number of Patients

Approximately 220 evaluable patients were enrolled.

### Treatment Beyond Disease Progression

Patients who tolerated the drug and were considered by the Investigator to be deriving clinical benefit will be permitted, with the Sponsor's approval, to continue with treatment beyond initial RECIST 1.1-defined progressive disease if they met the following clinical stability criteria:
Absence of clinical symptoms and signs (including worsening of laboratory values) indicating PD, i.e., patient is clinically stable per Investigator judgment.
No decline in Eastern Cooperative Oncology Group (ECOG) performance status attributed to underlying malignancy.
Absence of rapid progression of disease or progressive tumor at critical anatomical sites (e.g., cord compression) requiring urgent alternative medical intervention.

New lesions were considered measurable at the time of initial progression if the longest diameter was ≥10 mm (except for pathological lymph nodes, which must have a short axis of ≥15 mm). Any new lesion considered non-measurable at the time of initial progression became measurable and therefore included in the tumor burden measurement if the longest diameter increased to ≥10 mm (except for pathological lymph nodes, which must have increased in short axis to ≥15 mm).

### Treatment Duration

Patients received study treatment, until any disease progression (with exceptions noted as above), unacceptable toxicity, or patient wished to withdraw consent for any reason.

In Part 1, all patients in Cohorts A and B received botensilimab Q3W for up to 4 cycles (1 cycle=21 days) or approximately 3 months or until death, unacceptable toxicity, loss to follow-up, disease progression, withdrawal from the study, or the Sponsor closes the study, whichever occurs first.

In Part 2, all patients in Cohorts A and B received botensilimab Q3W for up to 4 cycles (1 cycle=21 days) or approximately 3 months in combination with balstilimab for up to 2 years (24 months) or until death, unacceptable toxicity, loss to follow-up, disease progression, withdrawal from the study, or the Sponsor closes the study, whichever occurs first.

After treatment completion/discontinuation (e.g., due to toxicity), patients were followed for safety at 30 days and 90 days, and for long-term follow-up every 3 months for at least 12 months from last administration. If the patient remained eligible to remain on the study despite study treatment completion or discontinuation they continued with scheduled imaging assessments and concurrent office visits.

### Prohibited Medications and Treatments

Medications or vaccinations specifically prohibited in the exclusion criteria were not allowed during the ongoing study. If there was a clinical indication for any medication or vaccination specifically prohibited during the study, discontinuation from study therapy or vaccination may have been required. The final decision on any supportive therapy or vaccination rested with the Investigator and/or the patient's primary physician. However, the decision to continue the patient on study treatment required the mutual agreement of the Investigator, the Sponsor, and the patient.

Listed below are specific restrictions for concomitant therapy during the course of the study:
Antineoplastic systemic chemotherapy or biological therapy.
Immuno-oncology therapies not specified in this protocol.
Investigational agents other than botensilimab and balstilimab.
Live vaccines.
Systemic glucocorticoids (>10 mg prednisone equivalent for >1 week) for any purpose other than to treat an immune-related adverse event (irAE). Note: Use of prophylactic corticosteroids to avoid allergic reactions (e.g., IV contrast dye or transfusions) was permitted, as was the use of inhaled steroids or intranasal or local injection of corticosteroids.

Patients treated with any prohibited medication (excluding the exceptions noted above) for clinical management were removed from the trial. All treatments that the Investigator considered necessary for a patient's welfare may have been administered at the discretion of the Investigator in keeping with the community standards of medical care. All concomitant medications were recorded on the electronic case report form (eCRF) including all prescription, over-the-counter products, herbal supplements, and IV medications and fluids. If changes occured during the trial period, documentation of drug dosage, frequency, route, and date was included on the eCRF. All concomitant medications received within 30 days prior to the first dose of study treatment and up to 30 days after the last dose of study treatment were recorded. Concomitant medications administered 30 days after the last dose of study treatment should have been recorded for SAEs as defined further below.

### Concomitant Medications

Medications taken by the patient during the study from the date of consent through the 30-Day Safety Follow-up Visit and the 90-Day Safety Follow-up Visit were recorded.

### Surgery and Radiation Therapy

If a patient required surgery for management of progressive malignant disease, they were discontinued from study treatment. In the instance of surgery for bowel obstruction, if there was no confirmed PD the patient may have continued receiving treatment on study. Palliative radiotherapy on non-target lesions may have been permitted following discussion with the Sponsor based on the specific clinical scenario.

### Permanent Discontinuation of Study Drug Treatment

Study drug administration (i.e., botensilimab) was permanently discontinued in a patient for any of the following:
Occurrence of an immune-related adverse event (irAE) that met the criteria for discontinuation.
Confirmed progressive disease unless the patient was considered by the Investigator to derive clinical benefit from the treatment, the patient was clinically stable, and there was approval from the Sponsor.
Clinical progression, in absence of radiologic progression on the basis of RECIST 1.1 as suggested by one or more of the following:
   Signs and/or symptoms consistent with clinically significant progression of disease, including worsening of laboratory values, appearance of new lesion(s)/worsening of the lesion best seen clinically, etc.
   Decline in ECOG performance status due to worsening malignancy.
   Tumor progression at critical anatomical sites that required urgent medical intervention, (e.g., CNS metastasis with potential for spinal cord progression).
Two or more consecutive doses of study therapy were missed due to non-compliance, unless otherwise approved by Sponsor.
Pregnancy

Tumor flare phenomenon, defined as local pain, irritation, or rash localized at sites of known or suspected tumor, did not require treatment discontinuation.

### Definition of End of Study

If the study was not terminated for a reason provided above, the overall trial ended 24 months after the last patient completed the 90 day safety follow-up visit; if the last patient withdrew from the trial, or was lost to follow-up (i.e., the patient was unable to be contacted by the Investigator) prior to the 90 day safety follow-up visit, the study will have ended approximately 24 months from the date of that event.

### B. Study Population

### Inclusion Criteria

In order to have participated in the study, a patient must have met all of the following inclusion criteria:

### Cohort A Only:

1. Prior treatment with anti-PD-(L) 1 at least 6 weeks, and radiologic progression confirmed by 2 scans at least 4 weeks apart, or if symptomatic due to progressive malignancy, then 1 scan showing progression was sufficient.
2. Prior progression must have been either on treatment with anti-PD-(L) 1 regimen or ≤12 weeks from last anti-PD-(L) 1 dose in metastatic setting or ≤24 weeks from completion of therapy in adjuvant/neoadjuvant setting.
3. For Part 2 only, no intervening anti-cancer therapy between the last course of anti-PD-(L) 1 treatment and the first dose of study treatment except for local measures (e.g., surgical excision, biopsy, focal radiation therapy), or BRAF+MEK inhibition when applicable in BRAF mutant patients.

### Cohort B Only:

1. Prior treatment with 1st generation anti-CTLA-4 (e.g., ipilimumab and tremelimumab), and prior treatment with anti-PD-(L)1 for at least 6 weeks.
2. Progression on most recent anti-neoplastic therapy.
3, For Part 2 only, no more than 3 prior lines of therapy in the advanced setting for BRAF mutation and no more than 2 prior lines of therapy in the advanced setting in the BRAF wild type.

### Cohorts A and B:

1. Voluntarily agreed to participate by giving signed, dated, and written informed consent prior to any study-specific procedures.
2. ≥18 years of age.
3. Histological confirmation of Stage III (unresectable) or Stage IV cutaneous melanoma, as per the AJCC 8th edition staging system.
4. Measurable disease on baseline imaging per RECIST 1.1 criteria.
5. BRAF V600 mutation status or consent to BRAF V600 mutation testing per local institutional standards during screening period.
6. Life expectancy≥3 months.
7. ECOG performance status of 0 or 1.
8. Adequate organ function defined as the following laboratory values within 21 days of Cycle 1 Day 1 (C1D1):
   a. Neutrophils>1500/μL (stable off any growth factor within 4 weeks of first study treatment administration).
   b. Platelets>100×10³/μL (transfusion to achieve this level was not permitted within 2 weeks of first study treatment administration).
   c. Hemoglobin>8.0 g/dL (transfusion to achieve this level was not permitted within 2 weeks of first study treatment administration).
   d. Creatinine clearance≥45 mL/min (measured or calculated using modification of diet in renal disease [MDRD]).
   e. Aspartate aminotransferase (AST)/alanine aminotransferase (ALT)<3.0×upper limit of normal (ULN).
   f. Total bilirubin<1.5×ULN, or <3.0×ULN for patients with Gilbert syndrome.
   g. Albumin≥3.0 g/dL.
   h. International normalized ratio or prothrombin time≤1.5×ULN and activated partial thromboplastin time≤1.5×ULN (unless patient was receiving anticoagulant therapy).
9. Patients must provide a sufficient and adequate formalin-fixed paraffin-embedded (FFPE) tumor tissue sample from the most recent biopsy of a tumor lesion, obtained within 90 days from signing informed consent form (ICF). If recent tumor tissue was unavailable or inadequate, a fresh biopsy was required, unless the Sponsor agreed that it is not safe/feasible.
10. Women of childbearing potential (WOCBP) must have a negative urine or serum pregnancy test at screening (within 72 hours of first dose of study medication) and prior to study drug administration. Non-childbearing potential was defined as:
   a.≥50 years of age and has not had menses for greater than 1 year.
   b. Amenorrheic for ≥2 years without a hysterectomy and bilateral oophorectomy and a follicle-stimulating hormone value in the postmenopausal range upon pre-study (screening) evaluation.
   c. Status was post-hysterectomy, bilateral oophorectomy, or tubal ligation. In Part 1, WOCBP must agree to use highly effective contraceptive measures starting with the screening visit through 90 days after the last dose of study treatment. In Part 2, WOCBP must agree to use highly effective contraceptive measures starting with the Screening Visit through 5 months after the last dose of study treatment. Highly effective contraception was defined in Appendix B, Guidance on Contraception, or as stipulated in national or local guidelines. Note: Abstinence was acceptable if this was the established and preferred contraception for the patient. WOCBP must have agreed not to donate eggs (ova, oocytes) during the treatment period and for at least 90 days after the last dose of study drug.
11. In Part 1, male patients with a female partner(s) of childbearing potential must have agreed to use highly effective contraceptive measures throughout the study starting with the screening visit through 90 days after the last dose of study treatment was received. In Part 2, male patients with a female partner(s) of childbearing potential must have agreed to use highly effective contraceptive measures throughout the study starting with the Screening Visit through 5 months after the last dose of study treatment was received. Males with pregnant partners must have agreed to use a condom; no additional method of contraception was required for the pregnant partner.
12. Was willing and able to comply with the requirements of the protocol.

### Exclusion Criteria

A patient was not enrolled in the study if any of the following criteria were present:

### Cohort A Only:

1. Received prior anti-CTLA-4 therapy.

### Cohort B Only:

1. Received prior Fc-engineered or Fc-enhanced anti-CTLA-4 therapy (e.g., BMS-96218, BMS-986288, HBM4003, XTX101, CTLA-4 targeting bispecific or other approaches such as ONC-392).

### Cohorts A and B:

1. Ocular, uveal, or mucosal melanoma.
2. Any persistent toxicities (Common Terminology Criteria for Adverse Events [CTCAE]≥2) from prior cancer therapy, excluding endocrinopathies stable on medication, stable neuropathy, and alopecia.
3. Any history of CTCAE≥3 immune-mediated toxicity (excluding endocrinopathies and non-necrotizing/bullous rash) from prior checkpoint inhibition.
4. Refractory ascites defined as requiring 2 or more therapeutic paracentesis in the last 4 weeks or ≥4 within the last 90 days prior to study entry.
5. Bowel obstruction or impending bowel obstruction within the past 3 months.
6. Clinically significant (i.e., active) cardiovascular disease: cerebral vascular accident/stroke or myocardial infarction within 6 months of enrollment, unstable angina, congestive heart failure (New York Heart Association class≥III), or serious uncontrolled cardiac arrhythmia requiring medication.
7. Active brain metastases or leptomeningeal metastases with the following exceptions:
   a. Treated brain metastases required a) surgical resection, or b) stereotactic radiosurgery. These patients must have been off steroids≥10 days prior to randomization for the purpose of managing their brain metastases. Repeat brain imaging following surgical resection or stereotactic radiosurgery was not required if their last brain MRI was within screening window. Whole-brain radiation was not allowed.
   b. Untreated isolated brain metastases that were too small for treatment by surgical resection or stereotactic radiosurgery (e.g., 1-2 mm) and/or of uncertain etiology were potentially eligible, but needed to be discussed with and approved by the study Medical Monitor.
8. Concurrent malignancy (present during screening) that required treatment or history of prior malignancy active within 2 years prior to the first dose of study treatment, i.e., patients with a history of prior malignancy were eligible if treatment was completed at least 2 years before the first dose of study treatment and the patient had no evidence of disease. Patients with history of prior early-stage basal/squamous cell skin cancer, low-risk prostate cancer eligible for active surveillance or noninvasive or in situ cancers who have undergone definitive treatment at any time were also eligible.
9. Incomplete resolution of clinical significant AEs related to most recent therapy/intervention prior to enrollment.
10. Severe acute respiratory syndrome coronavirus 2 (SARS-CoV-2) vaccine or booster<7 days before CID1. For vaccines requiring more than 1 dose, the full series should be completed prior to CID1, when feasible. Booster shot not required but also must be administered>7 days from CID1 or >7 days from future cycle on study.
11. Known allergy or hypersensitivity to any of the study drugs or any of the study drug excipients.
12. Any evidence of current interstitial lung disease (ILD) or pneumonitis or a prior history of ILD or non-infectious pneumonitis requiring high-dose glucocorticoids.
13. History of allogeneic organ transplant.
14. Psychiatric or substance abuse disorders that would interfere with cooperation with the requirements of the study.
15. Patients with a condition requiring systemic treatment with either corticosteroids (>10 mg daily prednisone equivalent) within 14 days or another immunosuppressive medication within 30 days of the first dose of study treatment. Inhaled or topical steroids, and adrenal replacement steroid doses>10 mg daily prednisone equivalent, were permitted in the absence of active autoimmune disease.
16. Active autoimmune disease or history of autoimmune disease that required systemic treatment within 2 years before starting treatment, i.e., with use of disease-modifying agents or immunosuppressive drugs.
17. History or current evidence of any condition, co-morbidity, therapy, any active infections, or laboratory abnormality that might confound the results of the study, interfere with the patient's participation for the full duration of the study, or is not in the best interest of the patient to participate, in the opinion of the treating Investigator.
18. A WOCBP who is pregnant or breastfeeding.
19. Previous SARS-CoV-2 infection within 10 days for mild or asymptomatic infections or 20 days for severe/critical illness prior to CID1.
20. Uncontrolled infection with human Immunodeficiency virus (HIV). Patients on stable highly active antiretroviral therapy (HAART) with undetectable viral load and normal CD4 counts for at least 6 months prior to study entry are eligible. Serological testing for HIV at screening is not required.
21. Known to be positive for hepatitis B virus (HBV) surface antigen, or any other positive test for HBV indicating acute or chronic infection. Patients who are receiving or who have received anti-HBV therapy and have undetectable HBV DNA for at least 6 months prior to study entry are eligible. Serological testing for HBV at screening was not required.
22. Known active hepatitis C virus (HCV) as determined by positive serology and confirmed by polymerase chain reaction (PCR). Patients on or who have received antiretroviral therapy were eligible provided they were virus-free by PCR for at least 6 months prior to study entry. Serological testing for HCV at screening was not required.
23. Dependence on total parenteral nutrition.

### C. Study Assessments and Procedures

It may have been necessary to perform these procedures at unscheduled timepoints if deemed clinically necessary by the Investigator. Furthermore, additional evaluations/testing may have been deemed necessary by the Investigator and/or the Sponsor for reasons related to patient safety.

In these cases, such evaluations/testing were performed in accordance with those regulations.

Adherence to the study design requirements was essential and required for study conduct.

All screening evaluations must have been completed and reviewed to confirm that potential patients meet all eligibility criteria. The Investigator maintained a screening log to record details of all patients screened and to confirm eligibility or record reasons for screening failure, as was applicable.

Procedures conducted as part of the patient's routine clinical management (e.g., blood count) and obtained before signing of the informed consent form (ICF) may have been utilized for screening or baseline purposes provided the procedure met the protocol-specified criteria and were performed within the time frame defined of other baseline assessments.

Repeat or unscheduled samples and images may have been taken for safety reasons or for technical issues with the samples.

### Screening

The Investigator, or qualified designee, must have obtained documented informed consent from each potential patient prior to participating in the clinical study and for optional genetic research.

After a patient signed an informed consent form (ICF), the patient will be assigned a unique, sequential patient number. Once a number was assigned, it was not reassigned if the original patient was found to be ineligible or withdrew consent.

Patients who fulfilled all the inclusion criteria and none of the exclusion criteria were enrolled into the study. Patients who did not meet the inclusion and exclusion criteria were considered screen failure, and their demographic information and reason for screen failure were documented.

During the screening period, attention was given to washout periods for prior treatments and prohibited medications.

### Treatment and Evaluation Period

Patients continued to receive all while they were actively receiving treatment.

### Treatment Discontinuation Visit

The discontinuation visit (≤7 days after criteria for permanent discontinuation was met) should occur only if study drug was discontinued for any reason other than completion of full study therapy duration (4 planned botensilimab cycles and/or up to 2 years of balstilimab treatment). If the discontinuation visit occurred approximately 30 days from the last dose of study drug, at the time of the mandatory 30-day safety follow-up visit, procedures were not repeated.

### 30-Day Safety Follow-Up Visit

The mandatory 30-Day Safety Follow-up Visit should be conducted for all patients 30 days (±7 days) after the last dose of study drug or before initiation of a new antineoplastic treatment, whichever comes first. Patients with an AE of Grade>1 will be further followed until the resolution of the AE to Grade 0 to 1 or until initiation of a new antineoplastic therapy, whichever occurs first. Patients with a treatment-related adverse event (TRAE) ongoing at the safety visit must be followed until the TRAE resolves, becomes stable, or is considered not clinically significant by the Investigator.

### 90-Day Safety Follow-Up Visit.

All patients who discontinue treatment for any reason will also have a 90-Day Safety Follow-up Visit (+7 days).

### Efficacy Follow-Up (Off Treatment, on Study)

Beginning at Week 18, patients will have visits coinciding with the imaging assessment schedule, Q9W through week 54 and then Q12W thereafter. Patients who discontinue study treatment for reasons other than progressive disease may continue with visits/imaging assessments on schedule.

### Survival Follow-Up (Off Study)

Patients with confirmed progressive disease, who have begun additional anti-neoplastic therapy, or who have discontinued the study for other reasons will continue to be followed for survival status Q12W (via telephone call) until the study ends. Survival data may be obtained from public records for patients who have been lost to follow-up.

### Post Study Anti-Cancer Therapy

The Investigator, or qualified designee, will review all new anticancer therapy initiated after the last dose of study drug. If a patient initiates a new anticancer therapy within 4 weeks after the last dose of study drug, the 30-Day Safety Follow-up Visit must occur before the first dose of the new therapy. Once new anticancer therapy has been initiated, patients will move into Survival Follow-up.

### Efficacy Assessments

Response assessment will be performed according to RECIST 1.1 [Eisenhauer 2009].

For all patients, tumor response assessment was obtained by radiographic imaging with computed tomography (CT [preferred]) or MRI (if CT is contraindicated) evaluation of the chest/abdomen/pelvis (plus other regions as required for specific tumor types or disease history). In general, lesions detected at baseline were followed using the same imaging methodology and preferably the same imaging equipment at subsequent tumor evaluation visits.

Tumor responses to treatment will be assigned based on evaluation of response of target, non-target, and new lesions according to RECIST 1.1 (all measurements should be recorded in metric notation) [Eisenhauer 2009]. To assess objective response, tumor burden at baseline was estimated and used for comparison with subsequent measurements. At baseline, tumor lesions were categorized in target and non-target lesions [Eisenhauer 2009].

Results for these evaluations were recorded with as much specificity as possible so that pre- and post-treatment results provided the best opportunity for accurately evaluating tumor response. Any complete response (CR) or partial response (PR) was confirmed at the next scheduled scan [Eisenhauer 2009]. If a patient discontinued treatment but remained on study, scans should be taken according to the treatment schedule. Additional imaging was performed if clinically indicated per the Investigator's discretion.

### Tumor Imaging

Initial tumor imaging was performed during the screening period to establish a baseline of disease burden. Scans performed as part of routine clinical management were acceptable for use as a screening scan if they were of adequate quality and ≤21 days prior to first dose.

The Investigator performed scans in addition to a scheduled trial scan if clinically indicated per the Investigator's discretion.

The timing of on-study imaging followed calendar days and was not be adjusted for delays in treatment administration or for visits. The same imaging technique was used in a patient throughout the trial for consistency.

### Brain Imaging

MRI, with or without contrast, was the preferred brain imaging modality; however, CT was acceptable if MRI was clinically contraindicated.

Patients with a history of central nervous system (CNS) metastases received brain imaging on the same schedule as the C/A/P imaging.

During the trial, brain CT/MRI scans were conducted if clinically indicated by development of new symptoms.

### Assessment of Safety

The safety profile of the study treatment was assessed through the recording, reporting, and analyzing of baseline medical conditions, AEs, physical examination findings, including vital signs, and laboratory tests. Comprehensive assessment of any apparent toxicity experienced by the patient was performed throughout the course of the study, from the time of the patient's signature of informed consent. Study site personnel reported any AE, whether observed by the Investigator or reported by the patient.

### Primary Efficacy Analyses

### Objective Response Rate (ORR)

ORR is defined as the proportion of patients who had best overall response (BOR) of objective responses (CR or PR). BOR is defined as the best response recorded from randomization (or start of the first dose in Cohort B patients) until data cutoff, disease progression, or the start of new anti-cancer treatment. Patients with no post-baseline response assessment were considered as non-responders for BOR. Confirmed ORR assessed by the Investigator per RECIST 1.1 was reported in each arm, as well as its corresponding Clopper-Pearson 95% CI. In addition, the ORR difference between arms (low dose vs high dose) within Cohort A was calculated with 95% CI constructed using Miettinen Nurminen method (Miettinen 1985). The study was not powered for hypothesis testing. Therefore, the lack of statistical significance can not be interpreted as evidence of no difference.

The proportion for each of the response categories (e.g., CR, PR, SD, PD, NE, and NA) will be presented.

ORR did not require confirmation and ORR in the per-protocol analysis was calculated in the sensitivity analysis.

The primary analysis will be performed approximately 6 months after the last patient has been randomized (or start of the first dose in Cohort B patients), and it will be considered as the final analysis of the study.

### Secondary Efficacy Analyses

### Progression-Free Survival (PFS)

PFS is defined as time from randomization (or start of the first dose in Cohort B patients) until progression assessed by investigator per RECIST 1.1 or death, whichever comes first. Patients without an event (death or progressive disease) at the analysis cutoff date will be censored on the date of last tumor assessment or start of new anti-cancer therapy. The median PFS and the cumulative probability of PFS at 6-month and 12-month will be calculated using Kaplan-Meier method for each treatment arm and presented with a two-sided 95% CI. The PFS censoring rule will follow the FDA Guidance for Industry Clinical Trial Endpoints for the Approval of Cancer Drugs and Biologics (Food and Drug Administration 2007). Data for patients without disease progression or death at the time of analysis will be censored at the time of the last adequate tumor assessment. Data for patients who are lost to follow-up prior to documented disease progression will be censored at the last adequate tumor assessment date when the patient is known to be progression free. Data for patients who start to receive new anti-cancer therapy will be censored at the last adequate tumor assessment date prior to the introduction of new therapy.

At final analysis, PFS distribution between treatment arms in Cohort A will be compared descriptively in a log-rank test. The hazard ratio between arms will be estimated from the Cox regression model.

### Duration of Response (DOR)

DOR will be analyzed as a time-to-event variable only in a subset of patients with response. All censoring rules for PFS analysis should be applied to DOR as well. DOR will be summarized using the Kaplan-Meier method. The median event time and 95% CI for the median will be provided using Brookmeyer and Crowley method (Brookmeyer 1982).

### Overall Survival (OS)

OS, defined as time to death of any cause. Patients will be censored either at the date that the patient was last known to be alive or the date of data cutoff, whichever comes earlier. The median OS and cumulative probability of OS estimated at 6-month intervals will be calculated using Kaplan-Meier estimates for each treatment arm and presented with a two-sided 95% CI.

Descriptive comparison of OS between arms in Cohort A will be made similarly as in PFS at the final analysis. The median OS time and 95% CI for the median will be provided.

### Safety Analyses

All Safety Endpoints were analyzed in the Safety Analysis Set, using actual treatment assignments, and pooling data from the 2 cohorts in the primary safety analysis.

### Study Medication Administration

Exposure to each treatment arm was summarized descriptively as the number of doses received, duration of exposure, dose intensity and relative dose intensity. Dose missed and dose interruption was also summarized.

### Adverse Events (AEs)

All AEs, both in terms of current MedDRA preferred term and NCI CTCAE 5.0 grade, were summarized descriptively by count (n) and percentage (%) for each treatment group. AEs observed up until 90 days following discontinuation of the study treatment (i.e., the last dose of randomized treatment) or until the initiation of the first subsequent anti-cancer therapy (including radiotherapy, with the exception of palliative radiotherapy) following discontinuation of study treatment (whichever occurs first) were used for reporting of all of the AE summary tables. This will more accurately depict AEs attributable to study treatment only, as a number of AEs up to 90 days following discontinuation of the study treatment are likely to be attributable to subsequent therapy.

The incidence of treatment emergent adverse events (TEAEs) were reported as the number (percentage) of patients with TEAEs by system, organ, class, and preferred term. The number (percentage) of patients with TEAEs was also summarized by relationship to the study drug. TRAEs included those AEs considered by the investigator to be related to a study drug or with missing assessment of the causal relationship. SAEs, deaths, TEAEs with Grade≥3 severity, irAEs, TRAEs and TEAEs leading to treatment discontinuation, dose interruption, or dose delay were summarized.

### D. Pharmacokinetics (PK)

Serum botensilimab and balstilimab PK parameters may include (but are not limited to) Cmaxₛₛ, Cminₛₛ, area under the drug concentration-time curve within time span t1 to t2 at steady state (AUC₍ₜ₁₋ₜ₂₎₋ₛₛ), area under the drug concentration-time curve from time zero to time t (AUC₍₀₋₁₎), area under the drug concentration-time curve from time zero to infinity (AUC₍₀₋ₛ₎), time to maximum observed drug concentration (tmax), elimination rate constant (λz), t_{1/2}, systemic drug clearance, and Vd.

Both NCA and compartmental modeling (e.g., PopPK) may be used to analyze PK. Additional PK exposure metrics for serum botensilimab and balstilimab will be assessed via PopPK.

### E. Immunogenicity

The immunogenicity assessment will be conducted to detect and measure ADAs against botensilimab and balstilimab, using multi-tiered strategy of screening, confirmatory and titer assays. The samples which are positive in ADA assay were tested for neutralizing antibodies. Blood samples for serum botensilimab and balstilimab immunogenicity/ADA were collected from patients at the designated timepoints. Blood samples may be used for additional bioanalytical characterization.

### F. Exploratory Biomarkers

Exploratory biomarkers may be assessed in fresh tumor biopsy samples.

An adequate FFPE tumor tissue sample, preferably from the most recent biopsy of a tumor lesion, collected either at the time of, or after, the diagnosis of advanced or metastatic disease has been made and from a site not previously irradiated must be available for biomarker assessments.

If no tumor tissue is available from within 90 days prior to signing an informed consent form (ICF), a fresh biopsy was required (if safe/feasible to obtain). If a fresh biopsy was not safe/feasible, and no tumor tissue was available from within 90 days prior to signing ICF, the Sponsor may request archival tissue>90 days old for research purposes.

If a tumor biopsy was obtained from a target lesion during eligibility assessment, it was preferred to obtain a new baseline scan. Biopsy of lesions on study was limited to non-target lesions or new lesions if their pathologic etiology is ambiguous.

Tissue processing: The tumor biopsy sample should be fixed in 10% neutral buffered formalin, embedded, and routinely processed for histological evaluation. Formalin substitutes are not suitable fixatives.

Provision of samples: If the tumor-containing FFPE tissue block cannot be provided in total, sections from this block should be provided that are freshly cut (within the last month), 5 μm thick, and mounted on positively charged slides. Preferably, 25 slides should be provided; if not possible, a minimum of 15 slides are required. SuperFrost Plus microscope slides and slide containers will be provided by the central lab.

The exploratory biomarker objective for this study is to identify and/or evaluate biomarkers that are:
Predictive of response to botensilimab±balstilimab therapy, i.e., predictive biomarkers.
Associated with progression to a more severe disease, i.e., prognostic biomarkers.
Associated with innate or acquired resistance to botensilimab±balstilimab therapy.
Associated with susceptibility to developing AEs or can lead to improved AE monitoring or investigation, i.e., safety biomarkers.
Indicators of botensilimab±balstilimab therapy activity (i.e., pharmacodynamic biomarkers) and support mechanisms of action.
Instrumental in improving our knowledge and understanding of disease biology and drug safety.

Both tissue and blood samples will be collected at screening and on treatment. Measurements will include but are not limited to CD16a allele polymorphisms (FcγRIIIa status), ctDNA (targeted panel), phosphorylated proteins and IHC staining of PD-L1 and other markers to characterize immune population in TME.

### G. Objectives and Endpoints

TABLE 4
Objectives and Endpoints

| | |
|---|---|
| Objectives (both cohorts) | Endpoints (both cohorts) |

Primary

| | |
|---|---|
| To evaluate the clinical efficacy | Objective response rate (ORR) as |
| of botensilimab as monotherapy | assessed by Response Evaluation |
| and in combination with | Criteria in Solid Tumors Version |
| balstilimab. | 1.1 (RECIST 1.1) criteria. |

Secondary

| | |
|---|---|
| To evaluate the clinical efficacy | Progression-free survival (PFS), |
| of botensilimab as monotherapy | defined as the time from enrollment |
| and in combination with balstilimab | until disease progression or death, |
| in delaying the time until disease | quantified as a rate at 6 and 12 months. |
| progression. | DOR, defined as the time from first |
| To evaluate the duration of | objective response until progression |
| response (DOR) in patients with an | of disease or death, quantified as |
| objective disease response to | median DOR. |
| botensilimab as monotherapy and in | OS time, defined as the time from |
| combination with balstilimab. | enrollment until death, quantified as |
| To evaluate the overall survival | median OS |
| (OS) of patients treated with | |
| botensilimab as monotherapy and | |
| in combination with balstilimab. | |

Safety

| | |
|---|---|
| To evaluate the safety and | Adverse events (AEs), serious adverse |
| tolerability of botensilimab as | events (SAEs), AEs leading to treatment |
| monotherapy and in combination | discontinuation or death. |
| with balstilimab. | |

Pharmacokinetics/Immunogenecity

| | |
|---|---|
| To characterize the | Serum botensilimab ± balstilimab |
| pharmacokinetic (PK) profile and | concentrations; serum botensilimab ± |
| immunogenicity of botensilimab as | balstilimab anti-drug antibodies (ADA). |
| monotherapy and in combination | |
| with balstilimab. | |

Exploratory

| | |
|---|---|
| To evaluate response(s) to | CD16A allele (FcγRIIIA) |
| botensilimab as monotherapy and in | polymorphisms, and other relevant FcγR |
| combination with balstilimab, based | polymorphisms. |
| on fragment crystallizable gamma | Biomarker endpoints may include, but |
| receptor SNPs (FcγR SNPs) affinity. | are not limited to analysis of the following |
| To evaluate potential biomarkers | potential markers related to mechanism of |
| of response, resistance, activity and | action or the disease under study; they |
| toxicity of botensilimab as | may be tested on tumor and/or peripheral |
| monotherapy and in combination | blood samples pre-, on and post-treatment: |
| with balstilimab. | Expression/presence of T-cell |
| | and natural killer (NK) cell |
| | markers, and markers of |
| | activation to characterize the |
| | tumor microenvironment, e.g., |
| | immunohistochemistry (IHC) |
| | assessments, programmed cell |
| | death protein (ligand) 1 (PD- |
| | [L]1) status. |
| | Circulating tumor |
| | deoxyribonucleic acid (ctDNA) |
| | levels and variants. |
| | Abundance and activation of |
| | cell surface receptor proteins |
| | and their downstream signaling |
| | pathways, e.g., phosphorylated |
| | proteins. |
| | Large scale genomic markers, |
| | e.g., whole exome sequencing |
| | and bulk ribonucleic acid |
| | sequencing (RNA-seq). |
| | Immune cell presence and |
| | activity, e.g., flow cytometric |
| | quantification and |
| | characterization of peripheral |
| | immune cell subtypes. |

Abbreviations: ADA: anti-drug antibodies; AE: adverse event; ctDNA: circulating tumor deoxyribonucleic acid; DOR: duration of response; FcγRIIIA: fragment-crystallizable gamma receptor IIIA; IHC: immunohistochemistry; NK: natural killer; ORR: objective response rate; OS: overall survival; PFS: progression-free survival; PK: pharmacokinetic(s); RECIST: response evaluation criteria in solid tumors; RNA-seq: ribonucleic acid sequencing; SAE: serious adverse event; TME: tumor microenvironment.

Cohort A (Part 1) includes a minimum of 60 advanced cutaneous melanoma patients (30 per arm). Patients were randomized 1:1 between Arms 1 and 2 (50 and 150 mg of botensilimab, respectively). There was no formal statistical comparison between arms. The sample size of 30 patients per arm allowed for 84% power of excluding an ORR of 10% (see, e.g., Pires et al. Lancet Oncol. 22 (6): 836-847 (2021); Zimmer et al. Eur. J. Cancer. 75:47-55 (2017); Idera Pharmaceuticals, retrieved from “ir.iderapharma.com/news-releases/news-release-details/idera-pharmaceuticals-announces-results-illuminate-301-trial” (2021)) by the lower bound of exact 90% CI, assuming a target ORR of 30% for each treatment arm. A nonbinding interim futility analysis of efficacy, with safety assessment, was performed for Cohort A without stopping enrollment approximately 9 weeks after the first 12 patients were enrolled on each treatment arm. Considering the limited amount of data available at the time of the interim futility analysis, it was based on unconfirmed responses. With ≤1 unconfirmed objective response among the first 12 patients of the 50 mg dose arm, the arm may be stopped. The criterion of ≤1 response among the first 12 patients of the 50 mg dose arm corresponds to ≤6% predictive probability of observing an ORR of at least 25% at the end of the study. The probability of ≤1 response among 12 patients is 88%, 66%, 28%, and 9% when true ORR is 5%, 10%, 25%, and 30%, respectively.

Cohort B (Part 1) also included a minimum of 60 advanced cutaneous melanoma patients (30 per arm). Patients were randomized 1:1 between Arms 1 and 2 (50 and 150 mg of botensilimab, respectively). No formal comparison between arms was made. This sample size allowed for 80% power to exclude an unacceptable ORR of 10% by the lower bound of exact 80% CI, assuming a target ORR of 25% of each treatment arm. A nonbinding interim futility analysis of efficacy, with safety assessment, was planned for Cohort B approximately 9 weeks after the first 10 patients were enrolled in each treatment arm. Considering the limited amount of data available at the time of the interim futility analysis, it will be based on unconfirmed responses. Any arm may be closed if there was no objective unconfirmed responses among the first 10 patients. The criterion of <1 objective response among 10 patients corresponds to ≤6% predictive probability of observing an ORR of at least 20% at the end of the study. The probability of no response among the first 10 patients is 60%, 35%, 11%, and 6% when true ORR is 5%, 10%, 20%, and 25%, respectively.

For Part 2, both Cohorts A and B included 50 patients assigned in each cohort. No formal statistical testing was planned. With 50 patients, the 95% CIs are estimated as (10.0%, 33.7%), (13.8%, 39.2%), (17.9%, 44.6%), (22.1%, 49.8%), (26.4%, 54.8%), and (35.5%, 64.5%) when the observed ORRs are 20%, 25%, 30%, 35%, 40%, and 50%, respectively. The lower bounds excluded targeted ORR in Part 1 (e.g., 20%-25%) when the observed ORR in the combination arm is ≥15% higher (e.g., 35%-40%); and they will exclude an ORR of 10% if the observed ORR is around the targeted ORR in Part 1 (e.g., 20%-25%).

### H. Initial Results

Initial analysis was performed to evaluate the best overall response (BOR), objective response rate (ORR), and disease control rate (DCR) in 10 efficacy evaluable cutaneous melanoma patients that received botensilimab as monotherapy or in combination with balstilimab, as described above. Specifically, 8 patients received botensilimab as monotherapy and 2 patients received botensilimab in combination with balstilimab. The 10 patients were anti-PD-1 (e.g., nivolumab) and/or anti-CTLA-4 (e.g., ipilimumab) relapsed/refractory (R/R) cutaneous melanoma patients-specifically, 2 patients that were anti-PD-1 R/R and 8 patients were anti-PD-1 and anti-CTLA-4 R/R.

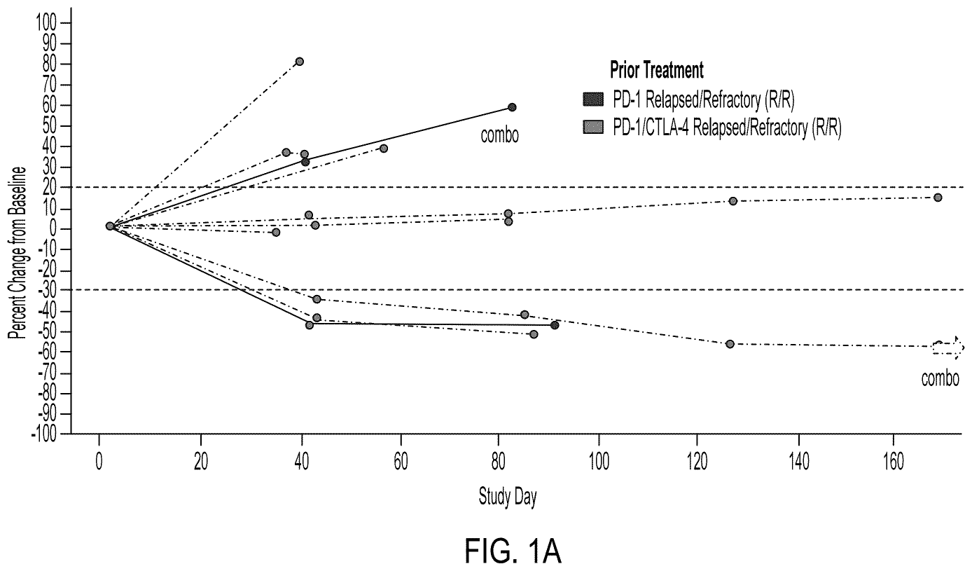

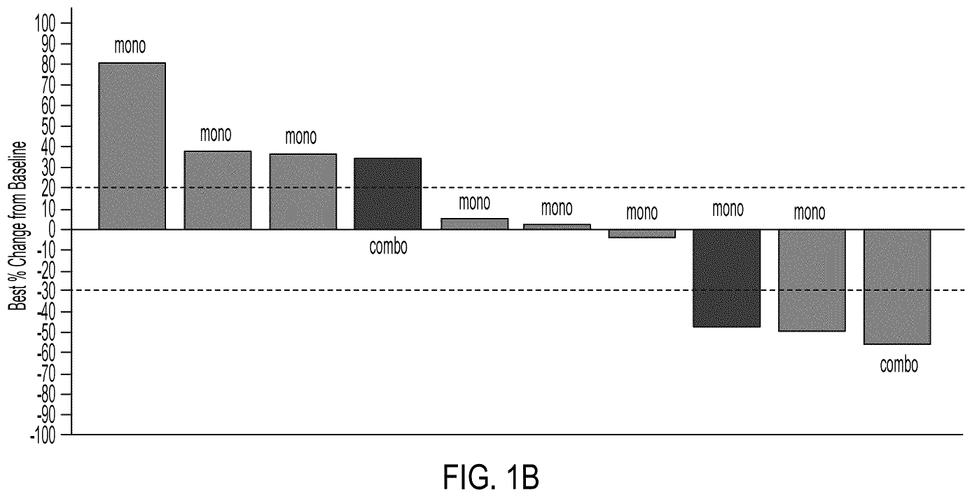

The percent change of tumor burden from baseline in the 10 patients over time, is shown in FIG. 1A, and the best percent change of tumor burden from baseline over time, is shown in FIG. 1B. BOR evaluation of these patients showed 0 patients with complete response (CR); 3 patients (30%) with partial response (PR); 3 patients (30%) with stable disease (SD); and 4 patients (40%) with progressive disease (PD). The DCR, defined as CR+PR+SD, was 60%. The ORR was 30%. Of the 10 patients, one out of two anti-PD-1 R/R patients showed a partial response, indicating a 50% ORR. Two out of eight anti-PD-1 and anti-CTLA-4 R/R patients showed a partial response, indicating a 25% ORR.

Botensilimab monotherapy responses were found to outperform conventional IgG1 (ipilimumab analog) in patients that express the low affinity FcγRIIIA V158F allele. See, e.g., Levey et al. (2022 Nov. 8-12). Botensilimab, a Novel Fc-Enhanced Anti-CTLA-4 Antibody Enhanced T Cell: APC Functionality and Promotes Superior Anti-Tumor Immunity [Poster presentation]. Society for Immunotherapy of Cancer, Boston, MA, United States, “agenusbio.com/wp-content/uploads/2022/11/Bot-preclinical_Levey_Chand_FINAL.pdf”, the entire disclosure of which is hereby incorporated by reference in its entirety. Based on the literature, response to ipilimumab is dependent on FcγRIIIA allele status and tumor mutational burden. About 40% of patients (i.e., those homozygous for the low affinity FcγRIIIA allele) have limited response with anti-CTLA-4 monotherapy (e.g., ipilimumab). See, e.g., Arce-Vargas et al. Cancer Cell. 33 (4): 649-663.e4 (2018). Unlike that reported for ipilimumab, responses to botensilimab were independent of FcγRIIIA allele status. Botensilimab monotherapy demonstrated a differentiated profile in cutaneous melanoma patients that are homozygous for the low (V/V) and high affinity FcγRIIIA allele (F/F), as well as in patients that are heterozygous (V/F) (Table 5). Table 5 shows BORR and BORR % data in Phase 1 and Phase 2 cutaneous melanoma patients that received botensilimab monotherapy, that were ipilimumab and nivolumab R/R.

TABLE 5
Botensilimab Monotherapy Responses

| | | | BRAF | FcγRIIIA | | | | |
|---|---|---|---|---|---|---|---|---|
| | Dose | | status | status | Prior Tx | BORR | BORR % | Sites |
| Phase | 2 | mg/kg | Mutant | V/V | 1. BRAF + | PR | −50% | Adrenal, |
| 1 | | | | | MEK | | | Inguinal, |
| | | | | | inhibitor | | | Oral/Cervical |
| | | | | | 2. anti-PD- | | | |
| | | | | | 1 | | | |
| | | | | | 3. anti- | | | |
| | | | | | CTLA-4 + | | | |
| | | | | | anti-PD-1 | | | |
| Phase | 150 | mg | Mutant | F/F | 1. anti-PD- | PR* | −65% | Liver, Lungs, |
| 2 | | | | | 1 | | | Soft Tissue |
| | | | | | 2. anti- | | | |
| | | | | | CTLA-4 + | | | |
| | | | | | anti-PD-1 | | | |
| | | | | | 3. BRAF + | | | |
| | | | | | MEK | | | |
| | | | | | inhibitor | | | |
| Phase | 150 | mg | WT | F/F | 1. anti-PD- | PR | −71% | Liver, Soft |
| 2 | | | | | 1 | | | Tissue |
| | | | | | 2. anti- | | | |
| | | | | | CTLA-4 + | | | |
| | | | | | anti-PD-1 | | | |
| Phase | 150 | mg | WT | F/F | 1. anti-PD- | PR* | −60% | Skin only |
| 2 | | | | | 1 | | | |
| | | | | | 2. anti- | | | |
| | | | | | CTLA-4 | | | |
| | | | | | 3. anti-PD- | | | |
| | | | | | 1 + anti- | | | |
| | | | | | LAG-3 | | | |
| Phase | 50 | mg | WT | F/F | 1. anti-PD- | PR* | −39% | Lungs, |
| 2 | | | | | 1 | | | Thoracic |
| | | | | | 2. anti- | | | Nodes |
| | | | | | CTLA-4 | | | |
| Phase | 50 | mg | WT | V/F | 1. anti-PD- | PR | −33% | Mediastinal, |
| 2 | | | | | 1 | | | Retroperitoneal |
| | | | | | 2. anti-PD- | | | & Inguinal |
| | | | | | 1 + anti- | | | Nodes |
| | | | | | CTLA-4 | | | |

### INCORPORATION BY REFERENCE

All patent and non-patent literature references cited above are incorporated herein by reference in their entirety.

1. A method of treating melanoma in a subject in need thereof, the method comprising administering to the subject an antibody that specifically binds to human Cytotoxic T-Lymphocyte Antigen 4 (CTLA-4) at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

2. A method of enhancing the activation of T cells in a subject who has melanoma, the method comprising administering to the subject an antibody that specifically binds to human CTLA-4 at a dose of 25 mg to 200 mg, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

3. The method of claim 1, wherein the antibody that specifically binds to human CTLA-4 is administered at a dose of 50 mg to 175 mg, 75 mg to 150 mg, 25 mg, 50 mg, 75 mg, 100 mg, or 150 mg.

4. (canceled)

5. (canceled)

6. The method of claim 1, wherein the antibody that specifically binds to human CTLA-4 is administered intravenously, optionally wherein the antibody that specifically binds to human CTLA-4 is administered by intravenous infusion over 30 minutes.

7. (canceled)

8. The method of claim 1, wherein the antibody that specifically binds to human CTLA-4 is administered once weekly, once every 2 weeks, once every 3 weeks, once every 4 weeks, once every 5 weeks, or once every 6 weeks.

9-13. (canceled)

14. The method of claim 1, wherein the antibody that specifically binds to human CTLA-4 is administered intravenously at a dose of 25 mg once every 3 weeks, 50 mg once every 3 weeks, 75 mg once every 3 weeks, 100 mg once every 3 weeks, or 150 mg once every 3 weeks.

15-18. (canceled)

19. The method of claim 1, wherein the dose is a therapeutically effective amount.

20. The method of claim 1, wherein;
the melanoma is cutaneous melanoma;
the melanoma is unresectable;
the melanoma is metastatic; and/or
the melanoma is refractory to a checkpoint inhibitor therapy, optionally wherein the checkpoint inhibitor therapy is an anti-PD-1 antibody, and anti-PD-L1 antibody, ipilimumab, or tremelimumab.

21-24. (canceled)

25. The method of claim 1, wherein the subject has received at least one prior anti-cancer therapy, optionally wherein:
a) the at least one prior anti-cancer therapy is an anti-PD-1 antibody; an anti-PD-L1 antibody; ipilimumab; or tremelimumab, further optionally wherein the anti-PD-1 antibody is balstilimab, nivolumab, or pembrolizumab; or
b) the at least one prior anti-cancer therapy is a BRAF inhibitor and/or a MEK inhibitor, further optionally wherein the BRAF inhibitor is selected from the group consisting of vemurafenib, dabrafenib, and encorafenib and/or the MEK inhibitor is selected from the group consisting of trametinib, cobimetinib, and binimetinib.

26-29. (canceled)

30. The method of claim 1, wherein the antibody that specifically binds to human CTLA-4 is administered to the subject prior to radiotherapy, a chemotherapy, or surgical removal of a tumor.

31. The method of claim 1, wherein;
the subject has not had prior anti-CTLA-4 antibody therapy;
the subject has unresectable Stage III or Stage IV cutaneous melanoma; and/or
the subject has a BRAF mutation, optionally wherein the BRAF mutation is a BRAF V600 mutation.

32-34. (canceled)

35. The method of claim 1, wherein;
the subject is homozygous for phenylalanine in position 158 of FcγRIIIA;
the subject is homozygous for valine in position 158 of FcγRIIIA; or
the subject is heterozygous for valine/phenylalanine in position 158 of FcγRIIIA.

36. (canceled)

37. (canceled)

38. The method of claim 1, wherein:
the melanoma is not ocular melanoma;
the melanoma is not uveal melanoma; and/or
the melanoma is not mucosal melanoma.

39. (canceled)

40. (canceled)

41. The method of claim 1, wherein;
1) the subject does not have any persistent toxicities (Common Terminology Criteria for Adverse Events [CTCAE]≥2) from prior cancer therapies;
2) the subject does not have any history of CTCAE≥3 immune-mediated toxicity (excluding endocrinopathies and non-necrotizing/bullous rash) from prior checkpoint inhibition;
3) administration of the antibody reduces tumor size in the subject;
4) administration of the antibody increases T-cell activation in the subject;
5) before administration of the antibody the subject has measurable disease on baseline imaging per RECIST 1.1;
6) before administration of the antibody the subject has an Eastern Cooperative Oncology Group performance status (PS) 0-1;
7) before administration of the antibody the subject has a predicted life expectancy of ≥3 months;
8) before administration of the antibody the subject has: adequate organ function as defined by one or more of:
a) neutrophils≥1500/μL;
b) platelets≥100× 10³/μL;
c) hemoglobin≥8.0 g/dL;
d) creatinine clearance≥45 mL/min as measured or calculated per local institutional standards;
e) AST/ALT≤3×upper limit of normal (ULN);
f) total bilirubin≤1.5×ULN (except patients with Gilbert syndrome who must have a total bilirubin level of ≤3.0×ULN);
g) albumin≥3.0 g/dL; and/or
h) International normalized ratio or prothrombin time≤1.5×ULN and activated partial thromboplastin time≤1.5×ULN (unless patient is receiving anticoagulant therapy);
9) the subject does not have partial or complete bowel obstruction within the last 3 months, signs/symptoms of bowel obstruction, or known radiologic evidence of impending obstruction;
10) the subject does not have refractory ascites defined as requiring 2 or more therapeutic paracentesis in the last 4 weeks or ≥4 within the last 90 days prior to administration of the antibody;
11) the subject does not have clinically significant cardiovascular disease;
12) the subject does not have active brain metastases or leptomeningeal metastases;
13) the subject does not have a concurrent malignancy that requires treatment or a history of prior malignancy that was active within 2 years prior to administration of the antibody;
14) the subject has not had a cytotoxic therapy or targeted therapy, within 3 weeks prior to administration of the antibody; and/or
15) the subject has not had other monoclonal antibody therapy, antibody-drug conjugate therapy, or radioimmunoconjugate therapy, within 4 weeks prior to administration of the antibody.

42-55. (canceled)

56. A method of treating melanoma in a subject in need thereof, the method comprising administering to the subject an antibody that specifically binds to human Cytotoxic T-Lymphocyte Antigen 4 (CTLA-4) at a dose of 50 mg once every 3 weeks or at a dose of 150 mg once every 3 weeks, wherein the subject has stage III or stage IV cutaneous melanoma, and wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

57. (canceled)

58. The method of claim 1, wherein the antibody that specifically binds to human CTLA-4 comprises the CDRH1, CDRH2, CDRH3, CDRL1, CDRL2, and CDRL3 amino acid sequences set forth in SEQ ID NOs: 1, 2, 3, 4, 5, and 6, respectively.

59. The method of claim 1, wherein the antibody that specifically binds to human CTLA-4 comprises a VH comprising the amino acid sequence set forth in SEQ ID NO: 7 and a VL comprising the amino acid sequence set forth in SEQ ID NO: 8.

60. The method of claim 1, wherein the antibody that specifically binds to human CTLA-4 comprises a human IgG1 heavy chain constant region comprising S239D/A330L/1332E mutations, numbered according to the EU numbering system.

61. The method of claim 1, wherein the antibody that specifically binds to human CTLA-4 comprises a heavy chain comprising the amino acid sequence set forth in SEQ ID NO: 9 and a light chain comprising the amino acid sequence set forth in SEQ ID NO: 10.

62. The method of claim 1, wherein the antibody that specifically binds to human CTLA-4 is botensilimab.

63-65. (canceled)


SEQUENCE LISTING (10 sequences)

SEQ ID NO: 1 - protein, 5 aa, synthetic construct
SYSMN

SEQ ID NO: 2 - protein, 17 aa, synthetic construct
SISSSSSYIYYAESVKG

SEQ ID NO: 3 - protein, 9 aa, synthetic construct
VGLFGPFDI

SEQ ID NO: 4 - protein, 11 aa, synthetic construct
RASQSVSRYLG

SEQ ID NO: 5 - protein, 7 aa, synthetic construct
GASTRAT

SEQ ID NO: 6 - protein, 9 aa, synthetic construct
QQYGSSPWT

SEQ ID NO: 7 - protein, 118 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 8 - protein, 107 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 9 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC